(19) United States
(12) Patent Application Publication
(10) Pub. No.: US 2003/0171720 A1
(43) Pub. Date: Sep. 11, 2003

(54) Syringe with rupturable bladder

(76) Inventor: Jesse Albert Pizolato, Coatesville, PA (US)

Correspondence Address: Jesse A. Pizolato, 110 Wilmont Circle, Coatesville, PA, 19320, US

(21) Appl. No.: 10/095,364

(22) Filed: Mar. 11, 2002

(51) Int. Cl. A61M 5/178

(52) U.S. Cl. 604/212; 134/22.1

(57) ABSTRACT

This invention relates to an improved syringe designed for effecting complete transfer of contents retained within the syringe. The improved syringe contains a rupturable fluid-filled bladder that is retained within the chamber of the syringe. When ruptured the bladder discharges the fluid retained therein and washes the interior of the chamber and component parts.

### BACKGROUND OF THE INVENTION

In the medical and veterinary fields such as invitro fertilization and reproductive medicine it is necessary to remove and transport microscopic cells including microscopic unfertilized eggs (blastocysts) and fertilized eggs (zygotes or embryos) that vary in size depending on their stage of development. Complete transfer of every blastocyst and embryo is universally desired because it assures the maximum number of developing structures to their respective sites, thereby increasing the probability of successful fertilization.

Currently, a threaded syringe (the Luer Lock System) that can be screwed to a pipette is used for the removal and transport of these microscopic structures during fertilization procedures. The operator of the syringe removes the structures (from where) by drawing back on the plunger and the microscopic cells are drawn into the pipette (aspiration). Then, they are transported and ejected from the syringe. After ejecting the microscopic cells from the pipette the doctor or laboratory technician must inspect the entire length of the pipette with the aid of a microscope to assure complete transfer of the microscopic cells. If any microscopic cells or components remain in the chamber of the syringe, e.g., the cells adhere to the walls of the pipette, the operator must aspirate a neutral fluid into the pipette again and attempt to free the cells from the walls of the pipette. This process must be repeated as often as necessary to assure thorough removal of all residual microscopic cells and effect complete transfer of all the cells to the appropriate environment.

Other cellular transfer procedures, whether they are medically or research related, require similar tedious examination and manipulation to assure that all cellular components reach the desired location.

The following patents show exemplary pre-filled syringes but none having a means for effecting complete removal of product such as microscopic cells from the chamber of the syringe.

U.S. Pat. No. 5,624,405 discloses a pre-filled syringe comprised of a glass body having a mouth portion including a tip for attachment of a needle. The body is filled with a medical solution. The tip member includes a top wall, a skirt portion and a tubular projection passing through the top wall and a covering means for closing the tubular projection. The unique tip member and covering allows for the economical use of glass as a syringe body for pre-filled syringes.

U.S. Pat. No. 5,554,125 discloses a pre-filled syringe for one or two component medicament. The syringe is comprised of a vial closed by piston. An adapter cap having an internal needle and an external connection for a needle is placed over a cap on the vial for turning it into a pre-filled syringe.

### BRIEF SUMMARY OF THE INVENTION

This invention relates to an improved syringe designed for effecting complete transfer of contents retained within the syringe and/or attached pipette. The basic syringe is comprised of a tubular chamber having a mouth for receiving liquids, e.g., pharmaceutical liquids, solutions containing microscopic cells, and the like into the chamber, a proximal end and a plunger inserted in the chamber from the proximal end for slidable and sealing engagement within the interior wall of the chamber. Fluids are introduced into the chamber, and/or attached pipette, on movement of the plunger away from the mouth and toward the proximal end by creating an internal negative pressure within the chamber. Conversely, fluids are discharged from the mouth due to increased internal chamber pressure when the plunger is moved away from the proximal end and toward the mouth. The improved syringe contains a rupturable fluid-filled bladder that is retained within the chamber. Often the fluid-filled bladder is both collapsible and rupturable and is attached to the plunger inside the chamber of the syringe.

There are significant advantages to the syringe described herein and these include:

an ability to “wash” the components introduced to the chamber and/or attachments of the syringe and thereby effectively remove all components therein;

an ability to economically provide a mechanism for washing the chamber and/or attachments of the syringe; and,

an ability to effect washing of the chamber and/or attachments of the syringe in one single step process.

### BRIEF DESCRIPTION OF THE DRAWINGS

FIG. 1 is a view in cross-section of a typical prior art syringe including a pipette that is commonly employed in transfer procedures.

FIG. 2 is a view in cross-section of the syringe of FIG. 1 including a rupturable bladder.

FIG. 3 is a view in cross-section of the syringe of FIG. 2 having residual fluid and microscopic cells retained in the chamber and pipette.

FIG. 4 is a view in cross-section of the syringe of FIG. 3 wherein the bladder has been ruptured and the fluid contents filling the mouth portion of the chamber and pipette.

### DETAILED DESCRIPTION OF THE INVENTION

The present practice of syringe washing during transfer procedures to assure complete transfer is commonly achieved by a multiple-step process. The improved syringe of this invention contains a rupturable bladder to facilitate the removal of contents introduced to the chamber of the syringe and/or any attachments to the syringe such as a pipette. To facilitate an understanding of the invention, reference is made to the drawings.

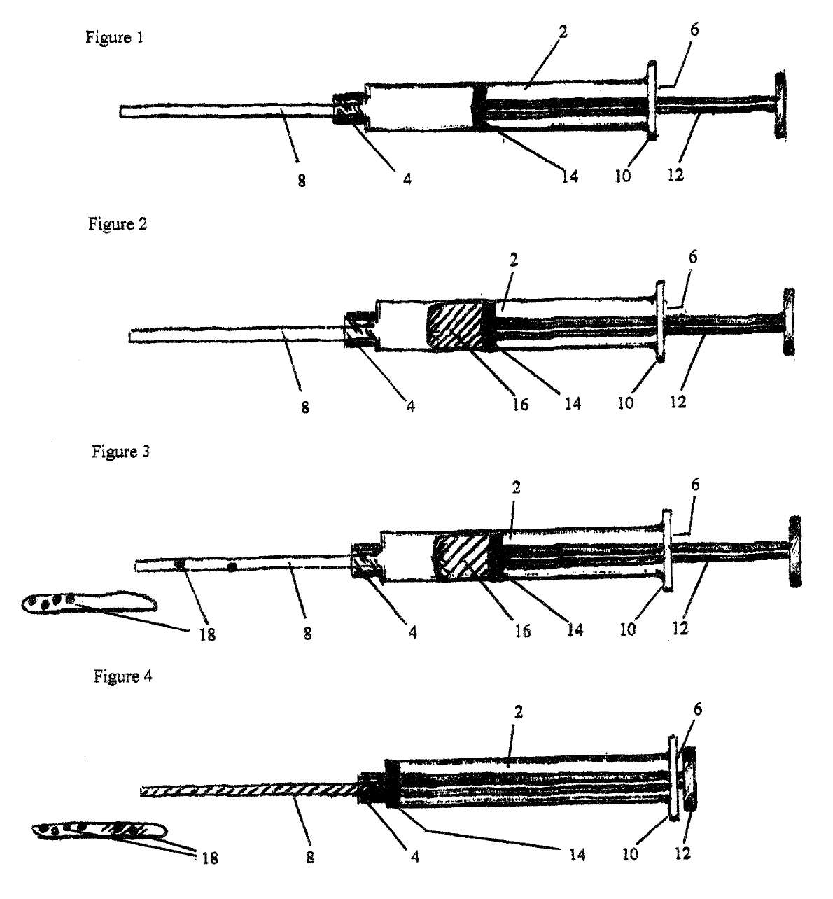

FIG. 1 is a view in cross-section of a syringe of a type commonly used in the art. It has a chamber 2, a mouth 4 for receiving fluid medicates, solutions containing microscopic cells, liquids and the like and a proximal end 6. The mouth 4 often is threaded (known as the Luer Lock Syringe) to receive a needle or pipette 8. The proximal end 6 is capped, typically by a flanged cap 10 thereby closing the chamber 2. The plunger terminates in a gasket 14 within the chamber 2 for effecting sealing engagement with the interior wall of the chamber 2. Liquids are then introduced to the chamber by inserting the needle 8 into a liquid and moving the plunger away from the mouth 4 and toward the proximal end 6, (negative internal chamber pressure). Ejection of the liquid from the mouth is effected by moving the plunger 12 from the proximal end 6 toward the mouth 4, (positive internal chamber pressure).

FIG. 2 is a view in cross-section illustrating the inclusion of a rupturable bladder 16 within the chamber 2. The bladder is comprised of a thin-walled polyethylene membrane, (or compartment) that bursts under moderate manual pressure as the plunger 12 is pushed toward the mouth 4. The design can possibly incorporate pin-like projections at the mouth 4 to lance the bladder and allow fluid release upon compression. Often the rupturable bladder 16 is appended to gasket 14. Other designs could incorporate the fluid-filled compartment within the plunger.

The rupturable bladder 16 carries a liquid solute, pharmaceutically acceptable to the material being transferred, and thereby provides a “built-in” washing capability. The neutral solute within the bladder is to have an inert effect upon the structures being transferred. As the components of cellular transfer procedures vary, the solute composition within the bladder can be modified by the manufacturer, e.g., pH, component osmolarity, viscosity, and actual chemical composition, to suit many respective cellular transfer requirements.

However, to assure complete washing and transfer of contents from the chamber and/or any attachments of the syringe, a quantity of solute slightly greater in volume than the internal volume of any attachment such as a pipette needs to be contained in the bladder. Often the volume can be from 1/16 to ⅜ of the volume of the chamber. Thorough ejection of components (thorough transfer) is accomplished by the displacement of aspirated liquid (containing cells) by a bladder solute upon ejection. Inadequate volume of bladder solute results in incomplete cellular displacement and consequently, incomplete cellular transfer.

Upon aspiration of the liquids from the chamber, e.g., solutions containing microscopic cells, the fluid-filled bladder ruptures and collapses against the interior of the mouth 4 thereby creating a flow of fluid that is propelled from the mouth and toward the opening of the pipette. This flow of fluid washes the walls of the pipette and consequently loosens any residual cells and carries all residual cellular structures to their desired location. If desired, the liquid solute in the rupturable bladder can be swirled within the chamber and then ejected by movement of the syringe toward the mouth.

The washing ability, and need thereof, is evidenced in FIG. 3 which shows the retention of microscopic cells 18 within the needle or pipette 8, after ejection of the liquid contents. FIG. 4 shows the mouth 4 of the syringe and needle or pipette 8 filled with a liquid such as a pharmaceutically acceptable carrier from the rupturable bladder.

The concept of providing a “washing” capability to syringes is not exclusive to fertilization procedures and can be applied to a multitude of transfer processes, as is common in stem cell research, where complete transfer is of paramount importance.

What is claimed is:

1. In a syringe comprised of a tubular chamber having a mouth for receiving liquids into the chamber, and a proximal end, a cap for sealing the proximal end said chamber, and a plunger inserted in the chamber from the proximal end for slidable and sealing engagement with the interior wall of the chamber, the improvement which comprises a rupturable fluid-filled bladder that is retained within the chamber whereby on discharge of the liquid from the chamber, the rupturable bladder is ruptured and thereby discharges the fluid retained therein.

2. The syringe of claim 1 wherein the fluid-filled bladder is both collapsible and rupturable.

3. The syringe of claim 2 wherein the rupturable bladder is attached to the plunger inside the chamber of the syringe.

4. The syringe of claim 3 wherein the volume of the rupturable bladder is from 1/16 to ⅜ of the internal volume of the chamber.
